(19) United States
(12) Patent Application Publication
(10) Pub. No.: US 2004/0054308 A1
(43) Pub. Date: Mar. 18, 2004

(54) Joint brace

(76) Inventors: Thorsten Herzberg, Hamburg (DE); Hugh Andrews, Koelln-Reisiek (DE); Stefan G. A. Bodenschatz, Buxtehude (DE); Richard G. Taylor, Cincinnati, OH (US)

Correspondence Address: KURT BRISCOE, NORRIS, MCLAUGHLIN & MARCUS, P.A., 220 EAST 42ND STREET, 30TH FLOOR, NEW YORK, NY, 10017, US

(21) Appl. No.: 10/362,398

(22) PCT Filed: Aug. 23, 2001
(86) PCT No.: PCT/US01/26416

(51) Int. Cl. A61F 5/00

(52) U.S. Cl. 602/21

(57) ABSTRACT

The specification and drawings describe and show an embodiment of the present invention in the form of a joint brace comprising:

a) a generally cylindrical portion of elastic material having a circumference C and a height H sufficient to embrace the joint of a user; and

b) a strap having a length L sufficient to wrap said strap about the joint of a user, said strap being joined on one end thereof to said generally cylindrical portion along a line parallel to a central axis of said generally cylindrical portion;

wherein the generally cylindrical portion comprises a portion comprising a stiffener, which when folded against a remaining portion of the generally cylindrical portion keeps the generally cylindrical portion from moving relative to the joint of the user while the strap is being wrapped around the joint of the user. In order to support the joint of a user, the generally cylindrical portion is placed around the joint; and the strap of said joint brace is wrapped about and secured about the joint of said user. It is emphasized that this abstract is provided to comply with the rules requiring an abstract which will allow a searcher or other reader to quickly ascertain the subject matter of the technical disclosure. It is submitted with the understanding that it will not be used to interpret or limit the scope or meaning of the claims.

This invention relates to a joint brace and the use thereof.

In the drawings, which illustrate an embodiment of the present invention, specifically, an embodiment wherein the joint brace is a wrist brace:

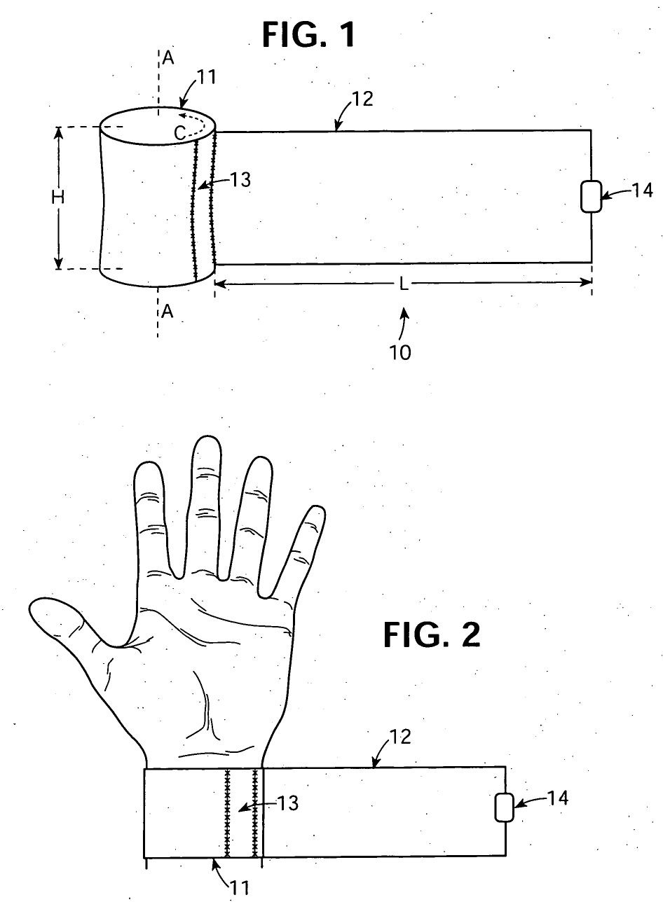

FIG. 1 is a perspective view of an example of such a wrist brace according to the present invention;

FIG. 2 is a drawing depicting the underside of the hand of a user showing the generally cylindrical portion of such exemplary wrist brace positioned over the wrist of the user;

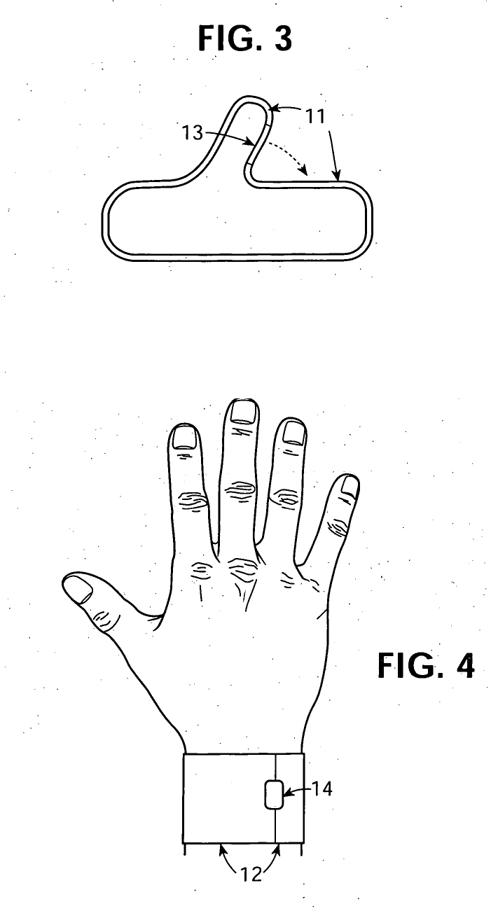

FIG. 3 is a drawing depicting a cross-section of the generally cylindrical portion of such exemplary wrist brace during the process of folding (indicated by the dashed line and arrow) one portion of the generally cylindrical portion, which comprises a stiffener, against a remaining portion of the generally cylindrical portion; and

FIG. 4 is a drawing depicting the topside of the hand of the user showing such exemplary wrist brace fully applied to the wrist of the user.

Referring now to the embodiment shown in the drawings, it can be seen that the exemplified wrist brace 10 comprises a generally cylindrical portion 11 attached at some point to strap 12. The generally cylindrical portion is made of elastic material and has a circumference C and a height H sufficient to embrace the wrist of a user. The generally cylindrical portion also has a central axis A, and the strap is joined to the generally cylindrical portion along a line substantially parallel to central axis A. The strap generally has a length L which is sufficient to wrap the strap about the wrist of a user in the manner as will be explained in greater detail hereinbelow. Both the generally cylindrical portion and the strap could be made out of neoprene, neoprene blends, woven or knit elasticized material. The neoprene material could be laminated with suitable textile products to provide the generally cylindrical portion with a soft lining to provide comfort.

On an end opposite that to which the strap is joined to the generally cylindrical portion, the strap comprises a first part 14 of a two-part fastener system. The strap also comprises on a surface thereof a second part of said two-part fastener system, so that said first part of said two-part fastener system can be fastened to said second part of said two-part fastener system to secure said strap about the wrist of said user. The first part of said two-part fastener system could comprise hook or loop fasteners, in which case, the second part of said two-part fastener system would comprise mating loop or hook fasteners.

Referring now to FIG. 2, the exemplified wrist brace is applied by slipping the hand through the generally cylindrical portion 11 so that the generally cylindrical portion becomes positioned over the wrist of the user. The generally cylindrical portion 12 comprises a sewn in, boxed portion 13, comprising a stiffener (not illustrated), which, as depicted in FIG. 3, is then folded against a remaining portion of the generally cylindrical portion 11, and acts to keep the generally cylindrical portion from moving relative to the wrist of the hand of the user while the strap is being wrapped around that wrist of the user. The stiffener could be of plastic, metal, paper or other suitable material, and can be positioned along the underside of the wrist to provide support thereto. As depicted in FIG. 4, the strap 12 is then wrapped around that wrist of the user and secured upon itself by means of fastener 14.

Although the foregoing embodiment relates to a wrist brace, the invention include braces for application to any body joint, for example, knees, ankles, elbows, etc., and, as with the described wrist brace, all such braces provide user adjustable compression to the joint being supported. In all of the inventive embodiments, the sizes of the generally cylindrical portion and the strap are a simple matter of design choice.

It should be understood that the preceding is merely a detailed description of one embodiment of this invention and that numerous changes to the disclosed embodiment can be made in accordance with the disclosure herein without departing from the spirit or scope of the invention. The preceding description, therefore, is not meant to limit the scope of the invention. Rather, the scope of the invention is to be determined only by the appended claims and their equivalents.

What is claimed is:

1. A joint brace comprising:
a) a generally cylindrical portion of elastic material having a circumference C and a height H sufficient to embrace the joint of a user; and
b) a strap having a length L sufficient to wrap said strap about the joint of a user, said strap being joined on one end thereof to said generally cylindrical portion along a line parallel to a central axis of said generally cylindrical portion;
wherein the generally cylindrical portion comprises a portion comprising a stiffener, which when folded against a remaining portion of the generally cylindrical portion keeps the generally cylindrical portion from moving relative to the joint of the user while the strap is being wrapped around the joint of the user.

2. The joint brace according to claim 1, wherein said strap comprises on an end opposite said one end a first part of a two-part fastener system and said strap comprises on a surface thereof a second part of said two-part fastener system, so that said first part of said two-part fastener system can be fastened to said second part of said two-part fastener system to secure said strap about the joint of said user.

3. The joint brace according to claim 2, wherein the first part of said two-part fastener system comprises hook or loop fasteners, and the second part of said two-part fastener system comprises mating loop or hook fasteners.

4. A method for supporting a joint of a user comprising:
a) providing a joint brace according to any one of claims 1-3;
b) placing the generally cylindrical portion around the joint of a user;
c) folding the portion comprising the stiffener, against a remaining portion of the generally cylindrical portion; and
d) wrapping the strap of said joint brace about the joint of said user.

5. The method according to claim 4, wherein said strap comprises on an end opposite said one end a first part of a two-part fastener system and said strap comprises on a surface thereof a second part of said two-part fastener system, and the method comprises fastening said first part of said two-part fastener system to said second part of said two-part fastener system to secure said strap about the joint of said user.

6. The method according to claim 5, wherein the first part of said two-part fastener system comprises hook or loop fasteners, and the second part of said two-part fastener system comprises mating loop or hook fasteners.
